(19) United States
(12) Patent Application Publication
(10) Pub. No.: US 2019/0175372 A1
(43) Pub. Date: Jun. 13, 2019

(54) Intravascular Device

(71) Applicants: Massachusetts Institute of Technology, Cambridge, MA (US); Washington University, St. Louis, MO (US)

(72) Inventors: Edward Stuart Boyden, Chestnut Hill, MA (US); Giovanni Talei Franzesi, Boston, MA (US); Christian Wentz, Cambridge, MA (US); Nir Grossman, Chestnut Hill, MA (US); Elazer R. Edelman, Brookline, MA (US); Colin Derdeyn, St. Louis, MO (US); Eric Leuthardt, St. Louis, MO (US)

(73) Assignees: Massachusetts Institute of Technology, Cambridge, MA (US); Washington University, St. Louis, MO (US)

(21) Appl. No.: 16/043,122

(22) Filed: Jul. 23, 2018

Related U.S. Application Data
(63) Continuation of application No. 14/681,076, filed on Apr. 7, 2015, now abandoned.
(60) Provisional application No. 61/976,498, filed on Apr. 7, 2014.

(51) Int. Cl. A61F 2/90; A61B 5/00; A61N 2/02; A61B 5/04; A61B 5/07; A61N 1/36; A61B 5/026

(52) U.S. Cl. CPC A61F 2/90; A61B 5/6862; A61N 2/02; A61B 5/04; A61N 1/375; A61N 1/3606; A61B 5/686; A61B 5/026; A61B 5/076

(57) ABSTRACT

An electronic intravascular device is placed in tight contact with vessel walls and is used for electrical stimulation and/or electrical recording of the vessel wall and surrounding target tissue. The electrodes may operate via connectors interfacing them to external hardware or may incorporate electronics to allow wireless power, information transfer, and control. The device includes an internal skeleton, a flexible substrate attached to the exterior of the skeleton, at least one pair of electrodes located on the substrate, and power and control circuitry connected to the electrodes. The power and control circuitry may include connectors for direct powering of the electrodes or circuit elements for wireless powering of the device by RF-based, optical-based, ultrasound-based, piezoelectric, or vibrational energy harvesting methods. The power and control circuitry may include circuit elements for wireless communication, including between the device and the external environment, and may include on-board processing for control of the electrodes.

### RELATED APPLICATIONS

This application is a continuation of U.S. patent application Ser. No. 14/681,076, filed Apr. 7, 2015, which claims the benefit of U.S. Provisional Application Ser. No. 61/976,498, filed Apr. 7, 2014, the entire disclosures of which are herein incorporated by reference.

### FIELD OF THE TECHNOLOGY

The present invention relates to electronic implantable devices and, in particular, to an electronic intravascular device.

### BACKGROUND

Electrical stimulation performed with implanted electrodes has emerged in recent decades as an extremely powerful clinical tool for the treatment of a variety of disorders, including, but not limited to, Parkinson's disease (Anderson 2006, Okun 2012), treatment-resistant depression (Kennedy 2011, Hoy 2010, Conway 2013), drug-resistant hypertension (Illig 2006, Heusser 2010), obesity (Dargent 2002), epilepsy (Jones 2010), and neuropathic pain (Nguyen 2011). More than 100,000 patients worldwide have so far been implanted with deep brain stimulation electrodes. Furthermore, electrical recordings obtained from human patients have shone new light on fundamental questions in neuroscience.

Procedures to implant current electrodes are typically invasive and, depending upon the target organ and region, may result in significant morbidity, both peri- and post-operatively (Beric 2001, Voges 2006, Goodman 2006). A promising alternative to existing strategies is to use the vasculature as a route, using routine (more than 600,000 procedures performed each year in the US alone (Chan 2011)) catheter-based methods to place stand-alone intravascular, intraluminal devices for stimulating and/or recording from a target tissue.

### SUMMARY

In illustrative implementations of this invention, an intravascular device is placed in tight contact with vessel walls and is used for electrical stimulation and/or electrical recording of the vessel wall and surrounding target tissue. The electrodes may operate either via thin connectors interfacing them to external hardware or may incorporate additional electronics to allow wireless power and information transfer and control.

In one aspect of the invention, an electronic intravascular device includes an internal skeleton, a flexible substrate attached to the exterior of the internal skeleton, at least one pair of electrodes located on the flexible substrate, and power and control circuitry connected to the electrodes and located on the flexible substrate. In some embodiments, the internal skeleton is a mesh stent. The power and control circuitry may include circuit elements for wireless powering of the device. Wireless powering may be RF-based, optical-based, ultrasound-based, piezoelectric, or adapted to perform vibrational energy harvesting. The power and control circuitry may alternatively include connectors for direct powering of the electrodes. The power and control circuitry may include circuit elements for wireless communication. The wireless communications circuitry may be RF-based, optical-based, or ultrasound-based. The circuit elements for wireless communication may be configured to allow communication between the device and the external environment. The circuit elements for wireless communication may include a power receive antenna and be configured to encode data by modulating the reflected impedance or absorbance of the power receive antenna. The power and control circuitry may include on-board processing for control of the electrodes. The electrodes may be configured for tissue stimulation, including electrical and/or magnetic tissue stimulation. The electrodes may be configured for recording data from the vascular wall, surrounding tissue, or both.

### BRIEF DESCRIPTION OF THE DRAWINGS

Other aspects, advantages and novel features of the invention will become more apparent from the following detailed description of the invention when considered in conjunction with the accompanying drawings wherein:

FIGS. 1A-C are top, cross sectional, and side view schematics, respectively, of an exemplary implementation of an intravascular device according to the invention;

FIGS. 2A and 2B are top, and cross sectional view schematics, respectively, of an alternative exemplary implementation of an intravascular device according to the invention;

FIG. 3 is a block diagram presenting a detailed view of an exemplary embodiment of a wireless power and control element according to one aspect of the invention, suitable for use in embodiments such as those depicted in FIGS. 1A-C and 2A-B;

FIGS. 4A and 4B are top, and cross sectional view schematics, respectively, of a finite element model used to support the development and testing of a novel trans-vessel neural interface according to an aspect of the invention;

FIGS. 5A-C are maps of exemplary electric field and current density distributions generated by an exemplary stimulating stent, depicting total electric field (Eₜₒₜₐₗ) (FIG. 5A), tangential electric field (E_{z}) (FIG. 5B), and radial electric field (Eₓ) (FIG. 5C) at the insulator plane, electrodes plane, and brain plane; and

FIG. 6 is a graph of exemplary total electric field (Eₜₒₜₐₗ) and total current density along a radial axis that that passes through the center of the electrode of an exemplary embodiment of the invention.

### DETAILED DESCRIPTION

An electronic intravascular device according to the invention is placed in tight contact with vessel walls and used for electrical stimulation and/or electrical recording of the vessel wall and surrounding target tissue. The electrodes may operate either via thin connectors that interface to external hardware or may incorporate additional electronics to enable wireless power and information transfer and control.

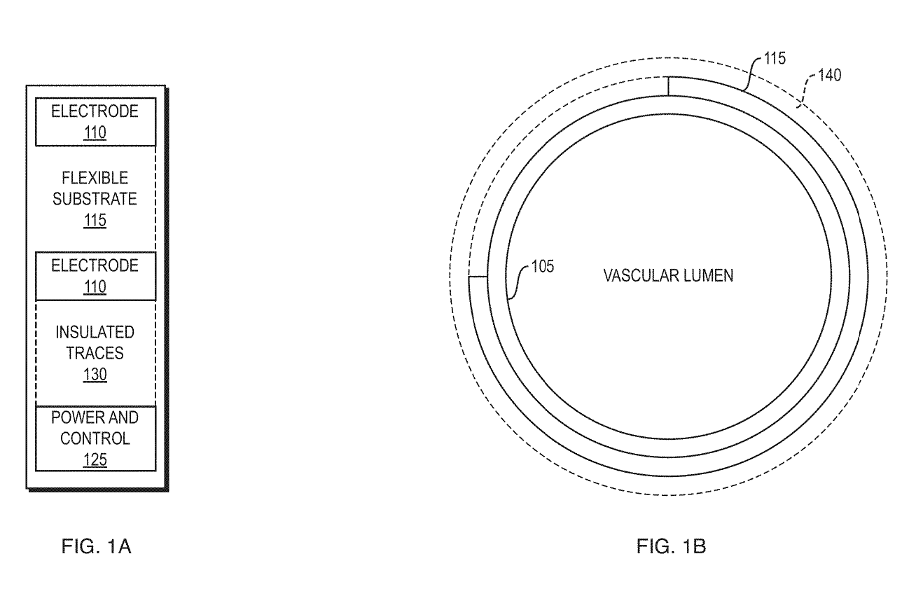

FIGS. 1A-C depict an illustrative implementation of an electronic intravascular device according to the invention. In the example shown in FIGS. 1A-C, the device comprises an internal skeleton 105, such as, but not limited to, a medical-grade mesh stent of dimensions appropriate to the target vessel, holding in place electrodes 110 of a suitable material (such as, but not limited to, platinum, platinum/iridium, or gold) and size (for example, but not limited to, 0.1-2 mm), patterned (such as, for example, but not limited to, with standard flexible PCB or microfabrication technology) on a thin (e.g. <250 um) flexible substrate 115 attached to the exterior of the stent. The device may be wire mesh or other metal, or may alternatively be a nonmetallic structural element, and may be coated or uncoated.

Electrodes 110 and wireless antenna(s) (optional) may be integrated into the stent structure. The electrodes may be powered either via connectors 120 (e.g. wires or patterned leads on the same flexible electrode board) or the electrode board may include additional circuit elements 125 for wireless (e.g. RF, ultrasound, piezoelectric) power delivery, using standard techniques known to those skilled in the art. This is connected to the electrode pads by insulated traces 130. Internal skeleton 105, once the device is implanted, ensures tight contact of electrodes 110 with vessel wall 140.

Although wire mesh stents in current clinical use typically have a diameter of 1.5-40 mm, the same technology can be used for vessels of substantially smaller diameter, and microfabricated versions of the active intravascular device can be made in sizes more than an order of magnitude smaller. For example, devices of only 5 um thickness may be fabricated from parylene-C and platinum, or other appropriate metals or combinations thereof. Such ultra-thin electrode boards may incorporate anchoring elements, be mounted on a suitable internal skeleton, or stresses within the device itself may be employed to give it the desired shape and ensure tight contact with the blood vessel wall.

In one exemplary implementation of an intravascular device according to the invention, 2 mm×1 mm bipolar electrodes, patterned on a 25 um polyimide surface, are attached onto a 5 mm OD stent. The connector, part of the flexible circuit board, allows interfacing to external hardware.

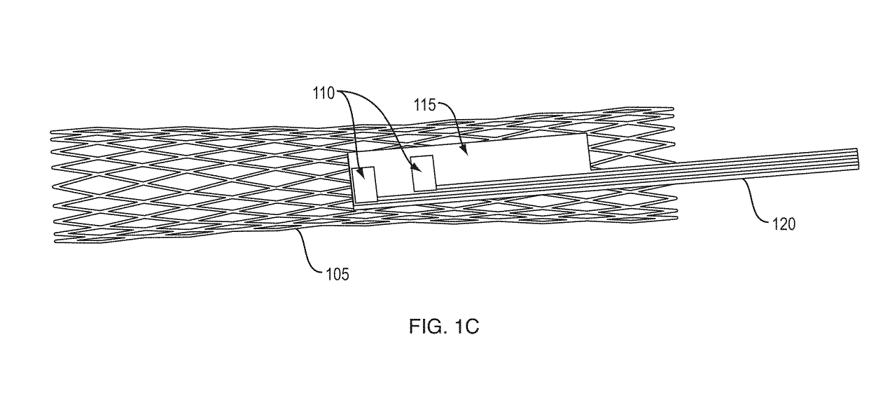

FIGS. 2A-B depict an alternative implementation of the active intravascular device. As seen in FIG. 2A, this embodiment is wirelessly powered and has on-board processer 205 for closed-loop control of electrodes 210, which can be used for both recording and stimulation. Two anchoring elements 215 are placed at the ends of the device, and can comprise, for example, metal meshes, two spring-like elements (metallic or polymeric) akin to spiral coil springs, or shape-memory pads. Anchoring elements 215 are kept in position by a sleeve over the implant during placement, but then relax and anchor the device to the vessel wall. Stiffening elements 220 further ensure tight contact with the wall. Before deployment, the active intravascular device in ‘wrapped’ configuration, can be mounted on a guide catheter (with or without a balloon, depending on the mechanism used by the anchoring elements) and protected by a retractable sleeve.

FIG. 2B depicts the exemplary device of FIG. 2A in cross section before deployment, with active intravascular device 230 in ‘wrapped’ configuration, mounted on a guidewire 235 with balloon 240 and protected by retractable sleeve 245.

In exemplary embodiments, the device can be fabricated in any way that enables tight contact between the electrodes/sensors and the vessel wall. For example, it can have an internal mesh skeleton supporting a flexible circuit overlaid on it, it may have smaller anchoring elements integrated within the circuit itself, or circuit elements, one or more antennas and electrodes, and structural support may be integrated as a homogeneous deployable unit.

The devices may be either passive or active, with capability for recording and/or stimulation, and several may be used together to create complex stimulation patterns. The devices may be powered and transmit data via connectors to external hardware or wirelessly, from/to an external unit. The device may, for example, receive power in the form of electromagnetic fields originating from another source, either external to the body or implanted, or it may harvest vibrational energy, either endogenous to the body (e.g. vessel wall pulsation), or delivered from an external source. It may also be optically powered. Similarly, readout may be achieved in a variety of ways, both passive and active, including RF-based, ultrasonic, and optical.

It will be clear to one of skill in the art that a device according to the invention may be powered in any of the many suitable ways known in the art, including RF-based wireless power, optical-based power, vibration harvesting, and direct connectors. In an illustrative embodiment of the device, the active intravascular device receives power wirelessly from electromagnetic fields originating from another source (e.g., an external transmit coil or array of coils worn on the head, neck, or body, depending on stent location, or implanted transmit coil(s), e.g. underneath the skull). In an illustrative embodiment, the stent receives power from one or more antennas placed orthogonal to one another. In an illustrative embodiment, the receive antenna is a dipole antenna deployed in the vasculature downstream of the body of the device.

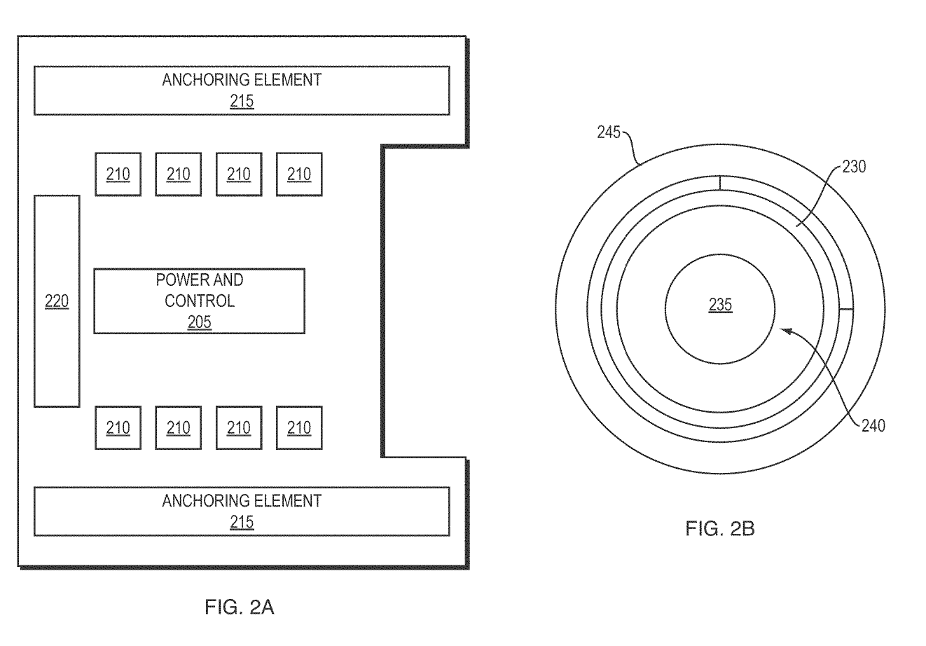

FIG. 3 is a block diagram depicting the basic elements of wireless power delivery and on-board control for the exemplary active stents of FIGS. 1A-C and 2A-B. The embodiment of FIG. 3 demonstrates electric/magnetic field based wireless power transfer using a resonant coupling between one or more transmit-side circuits and one or more implant-side circuits. (wireless power: vibration harvesting). Shown in FIG. 3 are power transmitting unit, which creates and transmits electromagnetic field 310 to implant-side power system 315, comprising receiving resonant coupling component 320, rectifier 325, and voltage conditioning unit 330.

In an alternative illustrative embodiment of the device, the intravascular active device is powered by harvesting tissue oscillations, either intrinsic (e.g. arterial vessel wall pulsation), or originating outside the body (e.g. with ultrasound, driven by an external transmitter coil or array of coils worn on the head or neck, depending on stent location, or by implanted transmit coil(s), e.g. underneath the skull).

In another alternative illustrative embodiment of the device, it may be optically powered by, for example, but not limited to, a photodiode or photodiode array.

In an illustrative embodiment, received signals are rectified and transiently stored on a capacitor to yield a DC voltage to be used for powering the rest of the device. The rectified DC voltage may additionally be converted to higher and/or lower voltages for operation of electronic subsystems. In an illustrative embodiment, the rectified voltage is stored on an ultracapacitor to transiently supply higher energy demands, e.g. for energizing stimulator electrodes, to operate communication systems, to operate sensor elements, to operate other electronic subsystems

The active intravascular device may have the capability to communicate with the external environment. The communication may be by, for example, but not limited to, RF, ultrasonic, or optical-based methods.

The active intravascular device of the invention can also be used to add chronic monitoring capabilities to existing stent interventional procedures. Application is not limited to the brain; it can be used, for example, but not limited to, for peripheral nervous system activation or general cardiovascular monitoring/control. Examples of parameters that may be monitored include, but are not limited to, endothelialization of the stent, flow through vessels, and continuous flow through the device.

The endothelialization of the stent may be chronically monitored via measurement of complex impedance, or by using spectroscopic methods.

By placing a pair of light sources (e.g. laser diodes) and pair of photodetectors (e.g. photo diodes) on either end of the stent, blood flow through the stent may be inferred. The first light source pulses light, which scatters off of objects flowing in the blood stream (e.g. red blood cells) and is detected at the first photodetector. The second light source similarly pulses light, which scatters off of the same object and is detected at second detector. Flow velocity is inferred as V=D/t.

By using a pair of pressure sensors (e.g., MEMS capacitive sensors) at either end of the stent, continuous flow through the stent may be monitored by comparing the difference in pressure between the two sensors, as P2−P1=Ra*V+Rb*V̂2, where Ra and Rb are empirically defined coefficients and V is the inferred velocity.

In an exemplary embodiment, the vascular device has circuitry onboard to measure small impedance values, e.g. for detection of pressure on a MEMS capacitive pressure sensor with single picofarad (pF) values and sub-pF variation across a pair of sensors. To measure these small values precisely an RC time constant detector is implemented, in which the MEMS capacitor is connected to a resistor and, as needed, additional reference capacitor. This RC tank is then charged up to an initial value, Vi. A comparator and timer circuit then measure the time it takes for this RC tank to discharge to a reference voltage, Vref. Using this RC time constant, the value of the MEMS capacitor is inferred. Additionally, to adjust for varying sized MEMS capacitors and to compensate for sensor aging, biofouling, etc., a bank of reference capacitors and resistors may be connected to the measurement RC tank to maintain viable time constant.

Active intravascular devices can stimulate tissue by electrical stimulation. The same type of devices may also be used for magnetic stimulation, which under certain circumstances can achieve better spatial selectivity, especially if using an array of coils. Also, this class of devices can be used as a ‘lens’ for an externally applied field. The circuit can also drive one or more light sources (e.g. laser diode or LED) for optical stimulation and control (e.g., optogenetic, optical uncaging, DREADDS, etc).

Neural structures beyond the immediate proximity of the vessel may be selectively activated by superposition of fields generated by a two or three dimensional arrangement of the active intravascular devices.

Miniature electromagnets can be used to generate time varying magnetic fields that penetrate the tissue with minimal deflection inducing more focal electric fields (eddy currents) at the neural tissue. A suitable arrangement of electromagnets (e.g. figure of 8) can be used to further focus and even steer the fields in space. Although these electromagnets may not carry large currents, their relative proximity to the cells and their small inductance (i.e. fast pulsing) may provide a very efficient stimulation.

In an illustrative embodiment, the reflected impedance of the power receive antenna(s) is modulated to encode data. An external antenna detects the change in receiver impedance, and converts the signal to data. In another example embodiment, the impedance of the transmit antenna is modulated to encode data. The stent detects the change in transmitter impedance, and converts the signal to data.

FIG. 3 depicts an illustrative embodiment of backscatter modulation, in which the impedance of the power receive antenna is modulated by applying a time-varying pulse pattern 350 to R_SW 355, which encodes data interpreted as time-varying matching impedance at power transmitting unit 305. Alternatively, the impedance of receiving resonant coupling component 320 may be modulated directly. Data may be similarly transmitted from power transmitting unit 305 to the intravascular device.

In an illustrative embodiment, a separate communications antenna is used to transceive data via electromagnetic waves to/from an external transceiver. In another illustrative embodiment, the absorbance/reflectance of the receiver antenna is modulated to encode data. In an illustrative embodiment, the intensity of the transmitted optical power to the active intravascular device is modulated to encode data. In another illustrative embodiment, the impedance of the receive transducer (ultrasonic power/data) is modulated to encode data.

The active intravascular devices can be placed either in a vein or artery. The decision for which vessel can depend on its proximity to adjacent structures, and/or the relative risk of placement. The intravascular device itself can be configured to match the caliber of the vessel when deployed or it can be configured to be slightly larger, to thus enable slow migration through the wall to allow for improved juxtaposition to adjacent target structures

The material of the portion of the intravascular device responsible for ensuring the tight juxtaposition of the active sites to the vessel wall can include coated or uncoated titanium, steel, NiTi and other shape-memory alloys, polymers, and in general any engineering material and composites with suitable mechanical characteristics, arranged in a suitable geometric configuration. The tight contact of the device to the vessel wall can be accomplished mechanically and/or chemically (e.g. with appropriate adhesive or using self-adhesive surfaces). The other flexible, nonconductive structural elements of the active intravascular device can be made of a suitable polymer (e.g. polyimide, kapton, parylene, etc.). The outer surface of electrode sites exposed to the body can be made of any suitable material, e.g. metals such as Pt, Pt/Ir, stainless steel, conducting polymers such as PEDOT or polypyrrole, carbon nanotubes, graphene and others as known to those skilled in the art.

In some embodiments, an active intravascular device may be used to record and transmit information about local electrical signals (especially for neural and cardiac application), recording and transmitting other information (e.g. strain, pressure, flow as inferred from two or more pressure sensors located at inlet and outlet of stent structure), as thin-film and/or MEMS sensors can be easily incorporated into the device. In addition, the devices can also be used to apply electrical fields, either to electrically stimulate a target tissue or to transiently disrupt (Hjouj et al. 2012) the blood-brain barrier (Ballabh 2004, Pardridge 2005) to allow temporally precise, highly localized delivery into the brain of drugs, nanoparticles etc. Moreover, the active intravascular devices can also be used to measure the electric properties of the surrounding tissue and of the internal blood. For example, measuring complex impedance using two or four electrodes configuration can identify changes in the tissue health, bleeding and process in the blood such as coagulation (thrombogenesis) in the blood.

In some embodiments, active intravascular devices can be used for applications of current extravascular implanted electrode systems. For example, some specific applications of active intravascular devices with stimulating electrodes include, with placement in the common carotid artery, stimulation of the baroreceptors in carotid sinus to control blood pressure. With placement in the internal carotid artery the vagus nerve can be stimulated for the control epilepsy, treatment of depression, reduce inflammation, facilitate recovery after a stroke, treatment of Alzheimer's, treatment of sleep apnea, and other clinical applications. Renal artery or renal vein placement allows stimulation to be used for the control of hypertension.

Moreover, an active intravascular device implanted in the base of the esophagus can be used to treat reflux. With placement in the coronary arteries active intravascular devices can be used to control atrial fibrillation. An active intravascular device placed in the gastric vein stimulation can mimic gastric electrical stimulation for obesity. An active intravascular device placed in the mesenteric vein can be used to stimulate the colon for the treatment of obesity.

By judiciously choosing the target location within the neurovasculature active intravascular device can be used in place of conventional DBS electrodes for the treatment of movement disorders, mood disorders, seizure disorders, Alzheimer's and neurodegenerative disorders, cognitive enhancement, tremor, spasticity, pain syndromes, Tourette's syndrome, headache, restless leg syndrome and other neurological derived diseases.

In light of the brain's high vascularization, it is in principle possible to target any desired location via vasculature-implanted devices of appropriate size. Among targets particularly suitable for vascular access are the anterior nucleus of the thalamus, the fornix, the nucleus accumbens, the subgenual cingulate white matter and the ventral caspule (Teplitzky et al. 2014). Suitable devices can be selected taking into consideration, among other factors, the diameter of the target vessel and its length within the region of interest, the minimum bend radius and number of branching points of the vessels transversed in reaching the target. Table 1 presents values for two representative target regions, nucleus accumbens and cingulate cortex, and relevant parameters for the selection of appropriate intravascular devices.

TABLE 1

| | | | | Min | |
|---|---|---|---|---|---|
| | | | Branches | bending | |
| | | Vessel | encountered | radius from | Length of this |
| Target | | size | from neck | neck to | vessel strand |
| region | Target vessel | (mm) | (ICA/IJV) | target (mm) | (mm) |
| Nucleus | ACA-A1 | 2.2 | 3 | 2-3 | 11 |
| accumbens | | | | (MCA-ACA | |
| | | | | branch) | |
| Cingulate | ACA-A2 | 1.8-2.2 | 3 | 2-3 | 8.7 |
| cortex | | | | (MCA-ACA | |
| | | | | branch) | |
| M1 | Superior Sagittal | 4.5-6 | ~7 | 18 | ~80 |
| | Sinus (and its | | | (Sigmoid | |
| | branches) | | | Sinus) | |
| S1 | Superior Sagittal | 4.5-6 | ~7 | 18 | ~80 |
| | Sinus (and its | | | (Sigmoid | |
| | branches) | | | Sinus) | |
| Posterior | Superior Sagittal | 4.3-6.3 | ~6 | 18 | ~40 |
| parietal | Sinus (and its | | | (Sigmoid | |
| cortex | branches) | | | Sinus) | |

An active intravascular device placed in the neural vasculature can be used to transiently, locally, and reversibly permeabilize the blood-brain barrier for local delivery of drugs, micro/nanostructures etc. This can be used, for example, to enhance pharmacologic effects for neuroactive drug regimens or can be used to enable better brain penetration of the brain parenchyma for chemotherapeutic regimens to treat brain cancers.

Intravascular devices capable of electrical recording and data transmission, with or without the ability to stimulate, may be used for prosthetic brain-machine interface applications. Suitable signals include single unit, multi-unit activity, local field potentials, and combinations thereof, as has been demonstrated with conventional extravascular implanted electrodes. Any region that can be used for this purpose employing conventional electrodes can also be a target for intravascular devices. Possible examples include the primary motor and somatosensory corteces, and the posterior parietal reach regions. Suitable devices may be chosen according to the criteria previously described. See Table 1 for data on example regions.

Other versions of the active intravascular device, incorporating electrodes for recording, electronics for wireless power transfer, transmission of signals to enable outside processing and diagnostics and electrodes for stimulation can be placed in IVC/SVC, in the coronary sinus or coronary artery as implantable electrophysiologic recording for closed-loop treatment. Such devices can be also used, implanted in the neural vasculature to record local field potentials, for the monitoring and treatment of seizures, migraines, depression, Alzheimer's. This can also be used as for a brain computer interface (BCI), as well as opening the door to novel closed-loop interventions for a variety of neurological and psychiatric disorders. Multiple active intravascular devices can be placed so as to comprehensively and selectively target a desired region or combination of regions. In addition to clinical applications, such active intravascular devices, with or without stimulation capabilities, may be used for basic research applications.

The active intravascular devices can also be used to measure the electric properties of the surrounding tissue and of the blood, for clinical or research applications. For example, measuring complex impedance using two or four electrodes configuration can identify changes in the tissue health, bleeding and processes in the blood such as coagulation (thrombogenesis). When combined with stimulation the coagulation properties of the blood can be altered for therapeutic effect. This can have important utility for treating lesions that require a reduction in blood flow, such as vascular tumors, arteriovenous malformations, pathologic vascular fistulas, and vascular injuries. This mechanism can enable controlled coagulation and thrombosis without the problem of the embolic agent migrating to distant or unintended vascular distributions.

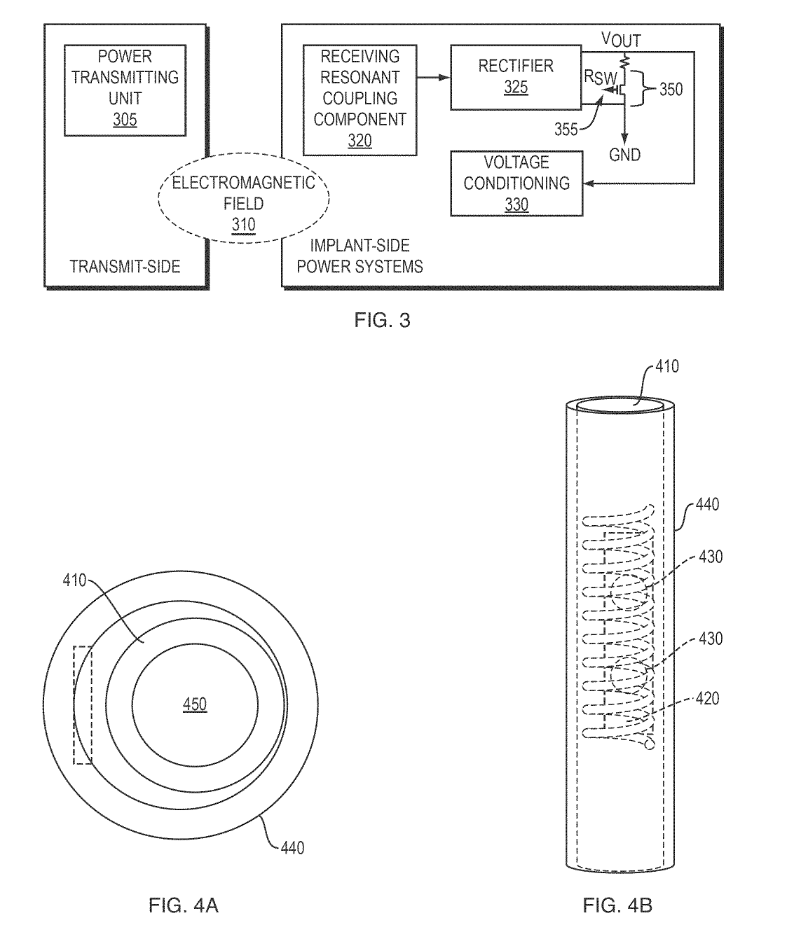

Finite element modeling was performed in order to support the development and testing of a novel trans-vessel neural interface. The modeling was done using the new SIM4LIFE platform from ZMT Zurich MedTech AG. FIGS. 4A-B show a schematic of the model consisting of stent 410, insulating sheet 420, electrodes 430, cylinder blood vessel 440, and flowing blood 450.

The tissue properties are based on the IT′IS Foundation online database of tissue properties. A resolution analysis was performed to ensure best grid. Since stimulation frequencies below 200 Hz (i.e. f<<1 MHz) are used, the model assumes a galvanic dominated current. In addition, as 2πfε<<σ, a galvanic dominated current is assumed. During the simulation, a voltage was applied between the electrodes and the total stimulation current was computed by summing the current density vectors over a virtual box around the electrodes. The current density distribution and the local resistivity of the tissue were used to calculate the specific absorption rate (W/m³). The model assumes convection heat transfer via blood flow with a rate equals to the temperature difference between the surface of the blood and the surface of the object touching it times a coefficient of 100.

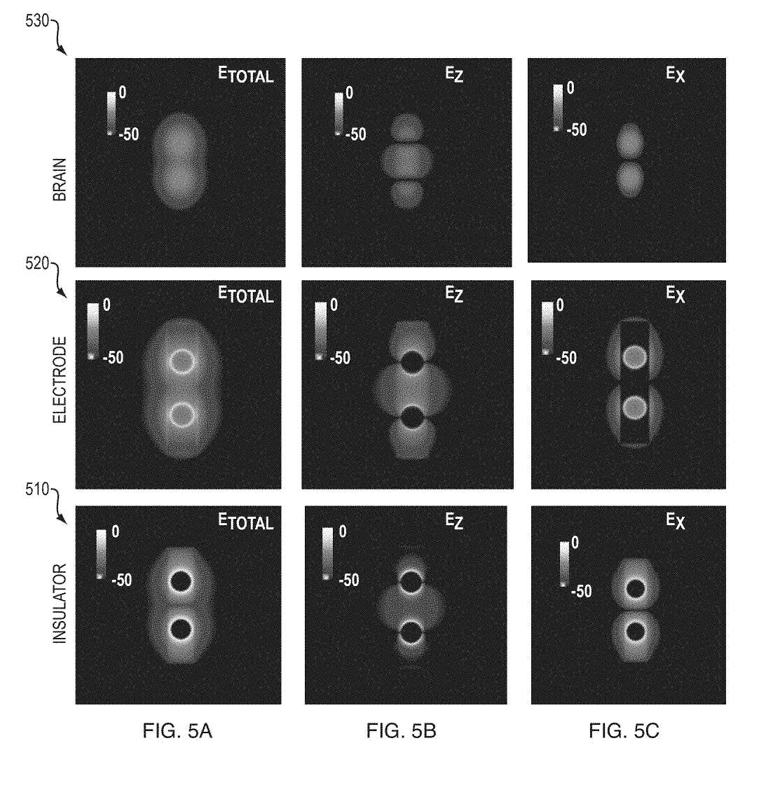

Results: FIGS. 5A-C show an example of the electric fields and current density distribution generated by the stimulating stent. In this example, a blood vessel model with an inner diameter of 3.5 mm, a wall thickness of 0.5 mm, and a length of 40 mm was used. The stent had a diameter of 6 mm and was 20 mm long. The stent was located at the center of the vessel. A 0.65 mm×17 mm insulating sheet with a thickness of 4 mm was placed above the stent. Two 0.5 mm diameter and 0.5 mm thick electrodes were located above the insulating sheet (i.e. between the stent and the vessel wall) at a 7 mm inter-electrodes spacing.

Seen in FIGS. 5A-C are maps of exemplary electric field and current density distributions generated by an exemplary stimulating stent, depicting total electric field (Eₜₒₜₐₗ) (FIG. 5A), tangential electric field (E_{z}) (FIG. 5B), and radial electric field (Eₓ) (FIG. 5C) at the insulator plane 510, electrodes plane 520, and brain plane 530 (i.e. outside the blood vessel).

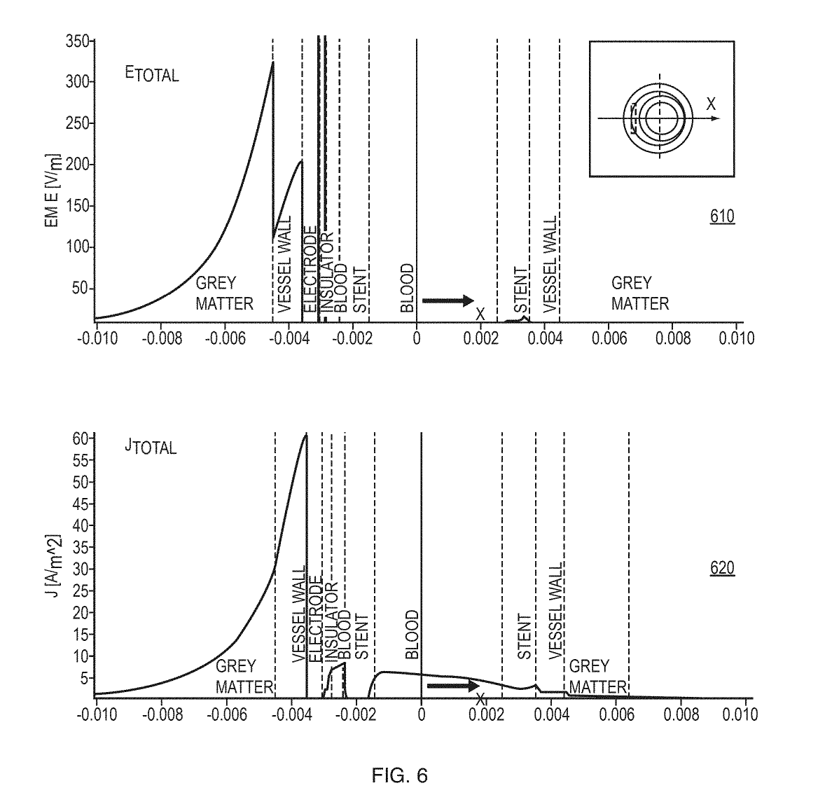

FIG. 6 is a graph of exemplary total electric field (Eₜₒₜₐₗ) 610 and total current density (Jₜₒₜₐₗ) 620 along a radial axis (x direction) that that passes through the center of the electrode.

Induced electric field at the brain region: A current of 1 mA through a pair of 3 mm diameter electrodes yields an approximately 25 V/m electric field at 5 mm distance from the vessel wall (i.e. inside the brain tissue). The field is only slightly lower (˜18 V/m) if there is a layer of blood between the electrode and the vessel wall. These fields magnitude are in principle sufficiently high to modulate neural activity (modulation threshold is approximately 1 V/m).

Distortion due to potential conductivity of the stent: An insulating sheet that is only slightly wider than the electrodes is sufficient to insulate the electrodes from a conductive stent, i.e. there is no need to coat the whole stent with an insulating material.

Edge effect: An edge can cause high density of current and hence local temperature rise. Edges in the shape of the electrode or due to a partial contact of the electrode with the vessel wall could lead to a high current density and a local rise in the tissue temperature. Thus, it is important to ensure a rounded electrode geometry and homogenous contact with the vessel.

Thermal risk: The overall temperature increase at the vessel and tissue is <0.3C° (the thermal increase due to the native metabolic processes of the brain), even in a case of a direct contact between the electrode and the vessel wall. The stent helps the blood to dissipate the heat. Interestingly, the stent can effectively cool the vessel below its normal temperature.

While several illustrative embodiments are disclosed, many other implementations of the invention will occur to one of ordinary skill in the art and are all within the scope of the invention. Furthermore, each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. A method for stimulation of a vascular wall and surrounding tissue, comprising:
inserting an electronic intravascular device into the vascular wall in a manner producing tight contact between electrodes on the device and the vascular wall, wherein the electronic intravascular device comprises:
an internal skeleton;
a flexible substrate attached to the exterior of the internal skeleton;
at least one pair of electrodes located on the flexible substrate, wherein the electrodes are configured for tissue stimulation; and
power and control circuitry connected to the electrodes and located on the flexible substrate; and
activating the electrodes to stimulate the vascular wall, the surrounding tissue, or both.

2. The method of claim 1, wherein the electronic intravascular device further comprises at least one anchoring element suitable for attaching the intravascular device to a vascular wall and the method further comprises the step of attaching the intravascular device to the vascular wall by the at least one anchoring element.

3. The method of claim 1, further comprising the step of recording parameters associated with at least one of the vascular wall, the surrounding tissue, or blood flowing within the device, wherein the electronic intravascular device comprises at least one pair of electrodes or sensor configured for recording data from at least one of the vascular wall, the surrounding tissue, or blood.

4. The method of claim 1, wherein the step of stimulating comprises at least one of electrical, magnetic, or optical stimulation.

5. The method of claim 1, wherein the intravascular device internal skeleton is a mesh stent.

6. The method of claim 1, wherein the intravascular device power and control circuitry comprises circuit elements for wireless powering of the device.

7. The method of claim 1, wherein the intravascular device power and control circuitry comprises connectors for direct powering of the electrodes.

8. The method of claim 1, wherein the intravascular device power and control circuitry comprises circuit elements for wireless communication configured to allow communication between the device and the external environment.

9. The method of claim 8, wherein the circuit elements for wireless communication comprise a power receive antenna and are further configured to encode data by modulating the reflected impedance or absorbance of the power receive antenna.

10. The method of claim 1, wherein the intravascular device power and control circuitry comprises on-board processing for control of the electrodes.

11. The method of claim 1, wherein the step of stimulating comprises employing a plurality of the electronic intravascular devices to create a complex stimulation pattern.

12. An electronic intravascular device specially adapted for carrying out the method of claim 1.

13. An electronic intravascular device specially adapted for carrying out the method of claim 2.

14. A method for electrical recording of parameters associated with a vascular wall and surrounding target tissue, comprising:
inserting an electronic intravascular device into the vascular wall in a manner producing tight contact between electrodes on the device and the vascular wall, wherein the electronic intravascular device comprises:
an internal skeleton;
a flexible substrate attached to the exterior of the internal skeleton;
at least one pair of electrodes or sensor located on the flexible substrate, wherein the electrodes or sensor are configured for recording data from at least one of the vascular wall, the surrounding tissue, or blood; and
power and control circuitry connected to the electrodes and located on the flexible substrate; and
recording parameters associated with at least one of the vascular wall, the surrounding tissue, or blood flowing within the device.

15. The method of claim 14, wherein the electronic intravascular device further comprises at least one anchoring element suitable for attaching the intravascular device to a vascular wall and the method further comprises the step of attaching the intravascular device to the vascular wall by the at least one anchoring element.

16. The method of claim 14, wherein the electronic intravascular device comprises at least one pair of electrodes located on the flexible substrate and configured for tissue stimulation, and further comprising the step of activating the electrodes to stimulate the vascular wall, the surrounding tissue, or both.

17. The method of claim 14, wherein the intravascular device internal skeleton is a mesh stent.

18. The method of claim 14, wherein the intravascular device power and control circuitry comprises circuit elements for wireless powering of the device.

19. The method of claim 14, wherein the intravascular device power and control circuitry comprises connectors for direct powering of the electrodes.

20. The method of claim 14, wherein the intravascular device power and control circuitry comprises circuit elements for wireless communication configured to allow communication between the device and the external environment.

21. The method of claim 14, wherein the circuit elements for wireless communication comprise a power receive antenna and are further configured to encode data by modulating the reflected impedance or absorbance of the power receive antenna.

22. The method of claim 14, wherein the intravascular device power and control circuitry comprises on-board processing for control of the electrodes or sensor.

23. An electronic intravascular device specially adapted for carrying out the method of claim 14.

24. An electronic intravascular device specially adapted for carrying out the method of claim 15.

25. An electronic intravascular device for stimulation and electrical recording of a vascular wall and surrounding tissue, comprising:
an internal skeleton;
a flexible substrate attached to the exterior of the internal skeleton;
at least one anchoring element attached to the intravascular device, the anchoring element being suitable for attaching the intravascular device to the vascular wall;
at least one pair of electrodes located on the flexible substrate, wherein the electrodes are configured for at least one of:
stimulation of at least one of the vascular wall or the surrounding tissue, and
recording data from at least one of the vascular wall, the surrounding tissue, or blood flowing through the device; and
power and control circuitry connected to the electrodes and located on the flexible substrate.

26. The intravascular device of claim 25, wherein the internal skeleton is a mesh stent.

27. The intravascular device of claim 25, wherein the power and control circuitry comprises circuit elements for wireless powering of the device.

28. The intravascular device of claim 25, wherein the power and control circuitry comprises connectors for direct powering of the electrodes.

29. The intravascular device of claim 25, wherein the power and control circuitry comprises circuit elements for wireless communication.

30. The intravascular device of claim 25, wherein the power and control circuitry comprises on-board processing for control of the electrodes.
